(19) United States
(12) Patent Application Publication
(10) Pub. No.: US 2023/0080234 A1
(43) Pub. Date: Mar. 16, 2023

(54) INFORMATION PROCESSING APPARATUS, INFORMATION PROCESSING SYSTEM, INFORMATION PROCESSING METHOD, AND COMPUTER PROGRAM

(71) Applicant: TERUMO KABUSHIKI KAISHA, Tokyo (JP)

(72) Inventors: Yoshihito MACHIDA, Sagamihara-shi (JP); Xiaowei LU, Hadano-shi (JP)

(73) Assignee: TERUMO KABUSHIKI KAISHA, Tokyo (JP)

(21) Appl. No.: 18/058,017

(22) Filed: Nov. 22, 2022

Related U.S. Application Data
(63) Continuation of application No. PCT/JP2021/019867, filed on May 25, 2021.

(30) Foreign Application Priority Data
May 25, 2020 (JP) 2020-090891

(51) Int. Cl. G16H 50/20; G06N 5/022

(52) U.S. Cl. CPC G16H 50/20; G06N 5/022

(57) ABSTRACT

An information processing apparatus includes a control unit configured to: determine, based on background information indicating one or more of a physical condition and a living environment of a patient, a symptom management method that includes one or more of: a management item to be managed at the time of managing a symptom of the patient, and a method for providing information to the patient; set a terminal device to executes a symptom management according to the symptom management method; acquire information indicating an implementation situation of the symptom management on the set terminal device; update, based on the implementation situation of the symptom management on the terminal device, the symptom management method according to which the terminal device executes the symptom management; and when the symptom management method is updated, set the terminal device to execute a symptom management according to the updated symptom management method.

### CROSS-REFERENCES TO RELATED APPLICATIONS

This application is a continuation of International Application No. PCT/JP2021/019867 filed on May 25, 2021, which claims priority to Japanese Application No. 2020-090891 filed on May 25, 2020, the entire content of both of which is incorporated herein by reference.

### TECHNOLOGICAL FIELD

The present disclosure relates to an information processing apparatus, an information processing system, an information processing method, and a computer program.

### BACKGROUND DISCUSSION

Regarding self-care of heart failure, there are various management indexes such as body weight, blood pressure, salinity (i.e., amount of salt in a given amount water), lower leg edema, orthopnea, exercise, appetite, dietary, fluid intake, and medicine-taking. As a method for recording information of a patient related to these management indexes, or as an educational material for providing medical information to a patient, a heart failure notebook in which a patient can physically record their heart condition and whose format can be unified for each hospital or region.

Japanese Patent Application Publication No 2015-64914 A describes a method of identifying a personal intervention which is available for a patient who has experienced at least one side effect related to a current intervention.

Japanese Patent Application Publication No. 2015-191469 A describes a dietary instruction support apparatus that supports an instructor in a dietary instruction for a dietary instruction subject.

Japanese Patent Application Publication No. 2016-508041 A describes a system in which a tailored list of monitoring parameters is selected based on user information related to a psychological profile of a user, and the user is monitored by using the list.

International Patent Application Publication No. WO 2019/035166 describes a treatment support apparatus that supports a treatment of a disease to be performed based on data acquired by a user sequentially using a system related to an information processing technique.

Not only the management indexes of the heart failure is diverse, but also pathological conditions and patient demographics of heart failure patients are various, and items or control ranges to be managed by a patient are different. Therefore, it requires extraordinary time and labor to manage and record all items using the heart failure notebook. Furthermore, since a co-morbidity, a cognitive function, a region characteristic, a family circumstance, a depression tendency and the like affect an educational effect and a level to be achieved, it is difficult to cope with individual patients with different backgrounds by the heart failure notebook whose format is unified. For example, a follow-up observation of jugular venous distention, which is a symptom index having very high sensitivity, can be performed by a patient having no problem in the cognitive function and having a high educational effect or a family member of the patient, but it is difficult for the patient or the like who has a symptom of reduced cognitive function (cognitive aging). These problems cannot be solved with the configurations of the published applications listed above.

### SUMMARY

The present disclosure is disclosed, which supports a symptom management matching a physical condition or a living environment of an individual patient.

An information processing apparatus according to an aspect of the present disclosure includes a control unit configured to: determine, based on background information indicating one or more of a physical condition and a living environment of a patient, a symptom management method that includes one or more of: a management item to be managed at the time of managing a symptom of the patient, and a method for providing information to the patient; set a terminal device such that the terminal device executes a symptom management according to the symptom management method; acquire information indicating an implementation situation of the symptom management on the set terminal device; update, based on the implementation situation of the symptom management on the terminal device, the symptom management method according to which the terminal device executes the symptom management; and when the symptom management method is updated, set the terminal device such that the terminal device executes a symptom management according to the updated symptom management method.

As one embodiment, the control unit sets the terminal device so as to execute the symptom management according to the symptom management method by transmitting information indicating the symptom management method to the terminal device.

As one embodiment, when an operation for updating the symptom management method is performed by a user, the control unit sets the terminal device such that the terminal device executes the symptom management according to the updated symptom management method.

As one embodiment, according to a rule indicating a relationship between the one or more of the physical condition and the living environment of the patient and the one or more of the management item to be managed at the time of managing the symptom of the patient and the method for providing information to the patient, the control unit determines the symptom management method based on the background information.

As one embodiment, for at least one sample patient, the at least one sample patient includes one or more of: a target patient that is the patient, and at least one another patient, the control unit acquires a prediction model which is created through machine learning by using, as training data, in a symptom management performed in the past for the at least one sample patient, one or more of background information of the at least one sample patient, a symptom management method of the symptom management performed in the past, and an implementation situation of the symptom management performed in the past, and a health condition of the sample patient after the symptom management performed in the past is performed; and when determining a symptom management method for the target patient based on the background information of the target patient, the control unit predicts a health condition of the target patient based on one or more of the background information of the target patient, the symptom management method for the target patient, and the implementation situation of the symptom management for the target patient by using the prediction model, and determines a symptom management method for the target patient based on the predicted health condition of the target patient.

As one embodiment, the background information includes information indicating one or more of a pathological condition, a co-morbidity, a cognitive function, a depression tendency, a region characteristic, and a family circumstance of the patient.

As one embodiment, the symptom management method includes one or more of a symptom management item, a method for inputting a symptom to the terminal device, a method for providing information from the terminal device, and an intervention method for the patient.

An information processing system according to an aspect of the present disclosure includes the above information processing apparatus; and the terminal device.

An information processing method according to an aspect of the present disclosure includes: determining, by a control unit, based on background information indicating one or more of a physical condition and a living environment of a patient, a symptom management method that includes one or more of: a management item to be managed at the time of managing a symptom of the patient, and a method for providing information to the patient; setting, by the control unit, a terminal device such that the terminal device executes a symptom management according to the symptom management method; acquiring, by the control unit, information indicating an implementation situation of the symptom management on the set terminal device; updating, by the control unit, based on the implementation situation of the symptom management on the terminal device, the symptom management method according to which the terminal device executes the symptom management; and when the symptom management method is updated, setting, by the control unit, the terminal device such that the terminal device executes a symptom management according to the updated symptom management method.

A non-transitory computer-readable medium (CRM) storing computer program code executed by a computer processor that executes a process comprising: determining, based on background information indicating one or more of a physical condition and a living environment of a patient, a symptom management method that includes one or more of: a management item to be managed at the time of managing a symptom of the patient, and a method for providing information to the patient; setting a terminal device such that the terminal device executes a symptom management according to the symptom management method; acquiring information indicating an implementation situation of the symptom management on the set terminal device; updating, based on the implementation situation of the symptom management on the terminal device, the symptom management method according to which the terminal device executes the symptom management; and when the symptom management method is updated, setting the terminal device such that the terminal device executes a symptom management according to the updated symptom management method.

According to the present disclosure, it is possible to support the symptom management matching a physical condition or a living environment of an individual patient.

### BRIEF DESCRIPTION OF THE DRAWINGS

FIG. 1 is a diagram illustrating a configuration of a system according to an embodiment of the present disclosure.

FIG. 2 is a block diagram illustrating a configuration of a symptom recording apparatus according to the embodiment of the present disclosure.

FIG. 3 is a block diagram illustrating a configuration of a symptom management apparatus according to the embodiment of the present disclosure.

FIG. 4 is a diagram illustrating an example of a screen of the symptom management apparatus according to the embodiment of the present disclosure.

FIG. 5 is a flowchart illustrating operations of the system according to the embodiment of the present disclosure.

FIG. 6 is a diagram illustrating an example of the screen of the symptom management apparatus according to the embodiment of the present disclosure.

FIG. 7 is a diagram illustrating an example of the screen of the symptom management apparatus according to the embodiment of the present disclosure.

FIG. 8A is a flowchart illustrating operations of the symptom management apparatus according to the embodiment of the present disclosure.

FIG. 8B is a flowchart illustrating operations of the symptom management apparatus according to the embodiment of the present disclosure.

FIG. 9A is a diagram illustrating an example of the screen of the symptom management apparatus according to the embodiment of the present disclosure.

FIG. 9B is a diagram illustrating an example of the screen of the symptom management apparatus according to the embodiment of the present disclosure.

FIG. 10A is a diagram illustrating an example of the screen of the symptom recording apparatus according to the embodiment of the present disclosure.

FIG. 10B is a diagram illustrating an example of the screen of the symptom recording apparatus according to the embodiment of the present disclosure.

FIG. 10C is a diagram illustrating an example of the screen of the symptom recording apparatus according to the embodiment of the present disclosure.

FIG. 10D is a diagram illustrating an example of the screen of the symptom recording apparatus according to the embodiment of the present disclosure.

### DETAILED DESCRIPTION

Set forth below with reference to the accompanying drawings is a detailed description of embodiments of an information processing apparatus, an information processing system, an information processing method, and a computer program. Note that since embodiments described below are preferred specific examples of the present disclosure, although various technically preferable limitations are given, the scope of the present disclosure is not limited to the embodiments unless otherwise specified in the following descriptions. In the drawings, the same or corresponding parts are denoted by the same reference numerals. In the description of the present embodiment, the description of the same or corresponding parts will be omitted or simplified as appropriate.

System Configuration

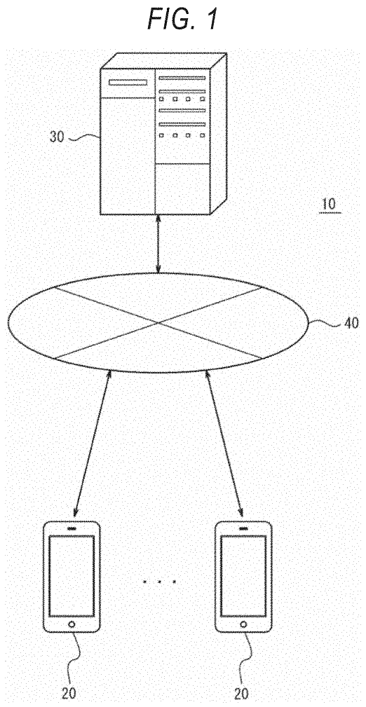

A configuration of a system 10 according to the present embodiment will be described with reference to FIG. 1.

The system 10 according to the present embodiment can be an information processing system that includes a plurality of symptom recording apparatuses 20 and at least one symptom management apparatus 30.

Each of the symptom recording apparatuses 20 can be an information processing apparatus (terminal device) used by a user such as a patient, a family member of the patient, or a caregiver. The patient can be, for example, a heart failure patient. The number of the symptom recording apparatuses 20 is not limited to a plural number, and may be only one. The symptom recording apparatus 20 is held by the user. Alternatively, the symptom recording apparatus 20 is set at home of the user. The symptom recording apparatus 20 can be, for example, a dedicated terminal such as a gadget, or a general-purpose terminal such as a mobile phone, a smartphone, a tablet, or a personal computer (PC). The symptom recording apparatus 20 can communicate with the symptom management apparatus 30 via a network 40.

The symptom management apparatus 30 is an information processing apparatus installed in a facility such as a medical institution or a data center. The symptom management apparatus 30 is implemented by a computer such as a PC or a server computer belonging to a cloud computing system or other computing systems, for example. In the present embodiment, an example of such a case in which the symptom management apparatus 30 is installed in the medical institution such as a hospital and is directly operated by a medical professional such as a doctor or a nurse will be described. However, the symptom management apparatus 30 may be installed in the data center or the like, and the medical professional may remotely operate the symptom management apparatus 30 via the network 40 by an information processing apparatus such as other PCs.

The network 40 includes the Internet, an intranet, at least one Wide Area Network (WAN), at least one Metropolitan Area Network (MAN), or a combination of the WAN and MAN. The network 40 may include at least one wireless network, at least one optical network, or a combination of the at least one wireless network and the at least one optical network. The wireless network can be, for example, an ad hoc network, a cellular network, a wireless Local Area Network (LAN), a satellite communication network, or a terrestrial microwave network.

Configuration of Symptom Recording Apparatus

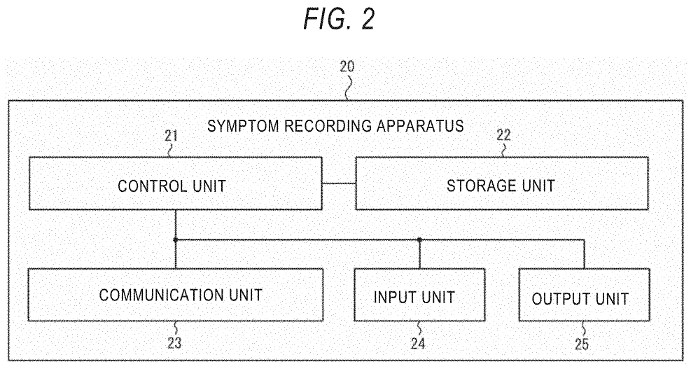

A configuration of the symptom recording apparatus 20 according to the present embodiment will be described with reference to FIG. 2. The symptom recording apparatus 20 can include a control unit 21, a storage unit 22, a communication unit 23, an input unit 24, and an output unit 25.

The control unit 21 can include at least one processor, at least one dedicated circuit, or a combination of the at least one processor and the at least one dedicated circuit. The processor is a general-purpose processor such as a Central Processing Unit (CPU) or Graphics Processing Unit (GPU), or a dedicated processor specialized for a specific process. The dedicated circuit can be, for example, a field-programmable gate array (FPGA) or an Application Specific Integrated Circuit (ASIC). The control unit 21 controls units of the symptom recording apparatus 20 to execute a process related to operations of the symptom recording apparatus 20.

The storage unit 22 can include at least one semiconductor memory, at least one magnetic memory, at least one optical memory, or a combination of at least two of these memories. The semiconductor memory can be, for example, a Random Access Memory (writable memory) (RAM) or a Read Only Memory (ROM). The RAM can be, for example, a Static Random Access Memory (SRAM) or a Dynamic Random Access Memory (DRAM). The ROM can be, for example, an Electrically Erasable Programmable Read Only Memory (EEPROM). The storage unit 22 can function as, for example, a main storage device, an auxiliary storage device, or a cache memory. Data used for the operations of the symptom recording apparatus 20 and data acquired by the operations of the symptom recording apparatus 20 are stored in the storage unit 22.

The communication unit 23 can include at least one communication interface. The communication interface can be, for example, an interface complying with a mobile communication standard such as Long Term Evolution (LTE), 4th Generation (4G), or 5th Generation (5G), an interface complying with short-range wireless communication such as Bluetooth®, or a LAN interface. The communication unit 23 receives the data used for the operations of the symptom recording apparatus 20, and transmits the data acquired by the operations of the symptom recording apparatus 20.

The input unit 24 can include at least one input interface. The input interface can be, for example, a physical key, an electrostatic capacitance key, a pointing device, a touch screen provided integrally with a display, a camera, or a microphone. The input unit 24 receives an operation of inputting the data used for the operations of the symptom recording apparatus 20. Instead of being provided in the symptom recording apparatus 20, the input unit 24 may be connected to the symptom recording apparatus 20 as an external input device. As a connection method, any method such as a Universal Serial Bus (USB), Wi-Fi®, a High-Definition Multimedia Interface (HDMI®), or Bluetooth® can be used, for example.

The output unit 25 can include at least one output interface. The output interface can be, for example, a display or a speaker. The display can be, for example, a Liquid Crystal Display (LCD) or an organic Electro Luminescence (EL) display. The output unit 25 outputs the data acquired by the operations of the symptom recording apparatus 20. Instead of being provided in the symptom recording apparatus 20, the output unit 25 may be connected to the symptom recording apparatus 20 as an external output device. As a connection method, any method such as USB, Wi-Fi, HDMI, or Bluetooth can be used, for example.

Functions of the symptom recording apparatus 20 are achieved by a processor serving as the control unit 21 executing a program (computer program) for the symptom recording apparatus 20 according to the present embodiment. That is, the functions of the symptom recording apparatus 20 can be achieved by software. The program causes a computer to execute the operations of the symptom recording apparatus 20, thereby causing the computer to function as the symptom recording apparatus 20. That is, the computer functions as the symptom recording apparatus 20 by executing the operations of the symptom recording apparatus 20 according to the program.

The program may be stored in a non-transitory computer-readable medium in advance. The non-transitory computer-readable medium can be, for example, a flash memory, a magnetic recording device, an optical disc, a magneto-optical recording medium, or a ROM. Distribution of the program can be executed by, for example, selling, transferring, or lending a portable medium such as a Secure Digital (SD) card, a Digital Versatile Disc (DVD), a Compact Disc Read Only Memory (CD-ROM) or a USB memory on which the program is stored. The program may be distributed by storing the program in a storage of a server in advance and transmitting the program from the server to another computer. The program may be provided as a program product.

For example, the computer temporarily stores, in a main storage device, the program stored in the portable medium, or the program transmitted from the server. Then, the computer reads, by the processor, the program stored in the main storage device, and executes, by the processor, a process according to the read program. The computer may read the program directly from the portable medium and execute the process according to the program. Each time the program is transmitted from the server to the computer, the computer may sequentially execute the process according to the received program. The process may be executed by a so-called Application Service Provider (ASP) type service in which the function is achieved only by execution instruction and result acquisition without transmitting the program from the server to the computer. The program includes data that is information provided for a process performed by an electronic computer and that is equivalent to the program. For example, data that is not a direct command to the computer but has a property that defines a process of the computer corresponds to “data that is equivalent to the program”.

A part of or all of the functions of the symptom recording apparatus 20 may be achieved by a dedicated circuit serving as the control unit 21. That is, a part of or all of the functions of the symptom recording apparatus 20 may be achieved by hardware. In addition, the symptom recording apparatus 20 may be implemented by a single information processing apparatus or may be implemented by cooperation of a plurality of information processing apparatuses.

Configuration of Symptom Management Apparatus

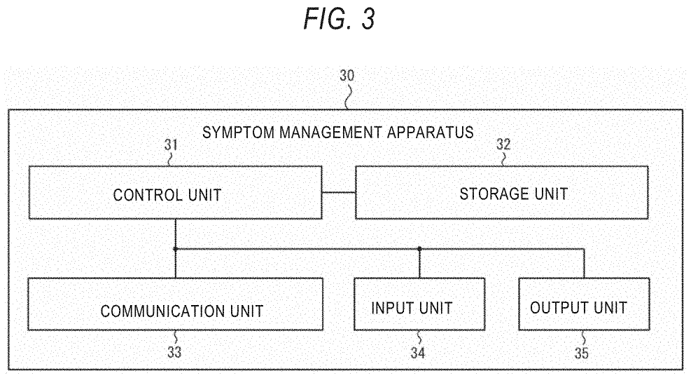

A configuration of the symptom management apparatus 30 according to the present embodiment will be described with reference to FIG. 3. The symptom management apparatus 30 can include a control unit 31, a storage unit 32, a communication unit 33, an input unit 34, and an output unit 35. The control unit 31, the storage unit 32, the communication unit 33, the input unit 34, and the output unit 35 are implemented by the same configurations as the control unit 21, the storage unit 22, the communication unit 23, the input unit 24, and the output unit 25 of the symptom recording apparatus 20, and achieve the same functions. Therefore, for the description of the configuration of the symptom management apparatus 30, the description of the configuration of the symptom recording apparatus 20 is incorporated by reference, and the details of configurations of the symptom management apparatus 30 and the symptom management recording apparatus 20 will be omitted.

Functions of the symptom management apparatus 30 can be achieved by a processor serving as the control unit 31 executing a program (computer program) for the symptom management apparatus 30 according to the present embodiment. That is, the functions of the symptom management apparatus 30 are achieved by software. The program causes a computer to execute operations of the symptom management apparatus 30, thereby causing the computer to function as the symptom management apparatus 30. That is, the computer functions as the symptom management apparatus 30 by executing the operations of the symptom management apparatus 30 according to the program. A method for providing the program and a method for executing the program are the same as those of the symptom recording apparatus 20, and thus the description of the symptom recording apparatus 20 is incorporated by reference, and the details of symptom recording apparatus 20 will be omitted.

A part of or all of the functions of the symptom management apparatus 30 may be achieved by a dedicated circuit as the control unit 31. That is, a part of or all of the functions of the symptom management apparatus 30 may be achieved by hardware. In addition, the symptom management apparatus 30 may be implemented by a single information processing apparatus or may be implemented by cooperation of a plurality of information processing apparatuses.

System Overview

In the symptom management apparatus 30, the control unit 31 determines, based on background information indicating at least one of a physical condition and a living environment of a patient, a symptom management method that includes at least one of: a management item to be managed at the time of managing a symptom of the patient, and a method for providing information to the patient. Further, the control unit 31 sets the symptom recording apparatus 20 such that the symptom recording apparatus 20 executes a symptom management according to the determined symptom management method. For example, the control unit 31 sets the symptom recording apparatus 20 so as to execute the symptom management according to the symptom management method by transmitting information indicating the symptom management method to the symptom recording apparatus 20 via the network 40. According to such a configuration, it is possible to support the symptom management according to individual backgrounds of the patient (for example, at least one of the physical condition such as a pathological condition, a co-morbidity, a cognitive function and a depression tendency of the patient, and the living environment such as a region characteristic and a family circumstance).

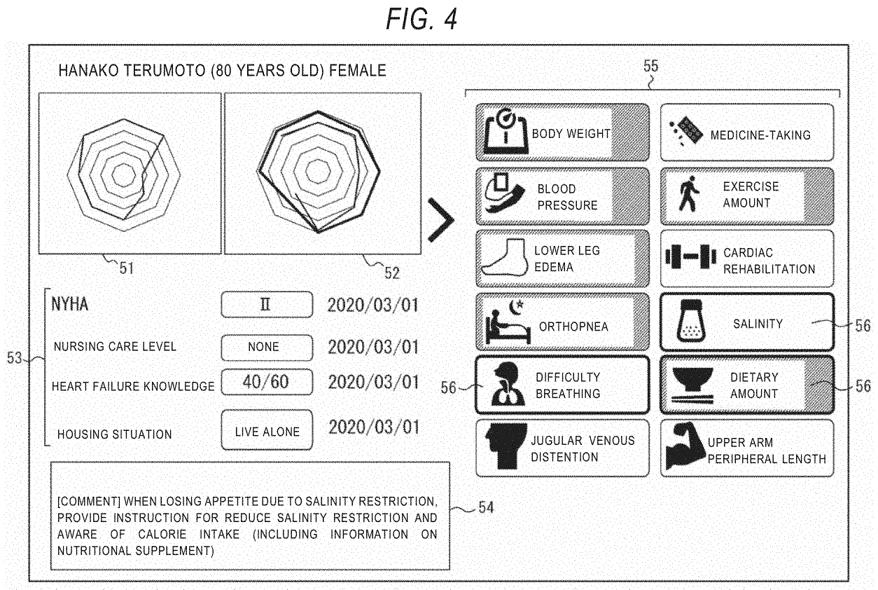

FIG. 4 is a diagram illustrating an example of a screen displayed on a display serving as the output unit 35 of the symptom management apparatus 30. FIG. 4 shows examples of background information 51 to 54 on a patient with a name “Hanako Terumoto”, an age “80 years old”, and a gender “female”, and symptom management items 55 determined based on the background information.

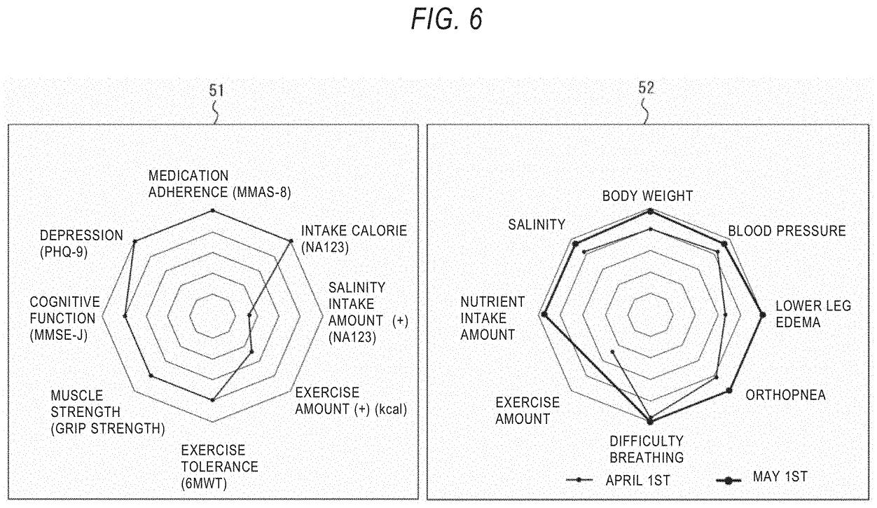

The background information 51 represents information investigated and measured in a medical institution with a line graph, and the background information 52 represents information investigated and measured by the patient with a line graph. These details will be described later with reference to FIG. 6. The background information 53 is an example of information investigated by a diagnostic inquiry or the like of a doctor or a nurse. In an example of the background information 53, a NYHA, a nursing care level of the patient, heart failure knowledge that the patient has known, and a housing situation (whether there is a housemate, or living-alone) are shown. The NYHA is a cardiac functional classification defined by New York Heart Association. The background information 54 is an example of text information indicating findings of the doctor, the nurse, or the like. The background information is not limited to the information illustrated here, and may include all kinds of information used for reference when determining the symptom management method, such as the pathological condition, the co-morbidity, the cognitive function, the depression tendency, the region characteristic, the family circumstance, and the like of the patient.

The control unit 31 of the symptom management apparatus 30 determines, based on the background information (may include information such as the age and gender of the patient) shown in the background information 51 to 54, the symptom management method that includes at least one of: the management items to be managed at the time of managing the symptom of the patient, and the method for providing information to the patient. The symptom management method may include at least one of a symptom management item, a method for inputting a symptom to the symptom recording apparatus 20, a method for providing information from the symptom recording apparatus 20 to the user, and an intervention method for the patient. The symptom management method can be classified into, for example, a maintenance item, a monitoring item, a management item, and an expression method. The maintenance item can include, for example, medicine-taking, a daily exercise amount (the number of steps, and the like), cardiac rehabilitation, resistance training, a daily activity, a vaccination, a drinking amount, and the like. The monitoring item can include, for example, a vital sign such as a body weight, a blood pressure, a body water content, and a heart rate, and symptoms such as lower leg edema, difficulty breathing, orthopnea, jugular venous distention, fatigability, depression, and cognitive function. The management item can include, for example, a potion instruction, a temporary rest, a dietary change, a delivery of functional nutritional food, an additional check instruction, a consultation recommendation, a family member alert, a caregiver alert, a health care worker alert, and the like. The expression method can include, for example, switching and a content of a still image/moving image/text/voice, and a language (including dialect).

The symptom management items 55 are examples of the symptom management item determined based on the background information 51 to 54. In the examples of the symptom management items 55, items of the “body weight”, the “blood pressure”, the “lower leg edema”, the “orthopnea”, the “difficulty breathing”, an “exercise amount”, and a “salinity” are determined as subjects of management by the control unit 31. However, by an expert user such as a doctor, the terms “difficulty breathing” and “salinity” indicated by 56 are excluded from the management items, and a term “dietary amount” can be added to the management items. As described above, when an operation for updating the symptom management method is performed by the user, the symptom recording apparatus 20 can be set so as to execute a symptom management according to the updated symptom management method rather than setting the determined symptom management method to the symptom recording apparatus 20 as it is. Accordingly, it is possible to perform a more appropriate symptom management by reflecting the determination of the user such as a doctor in the selection of the symptom management method.

When the symptom management method including such symptom management items 55 is determined, the control unit 31 of the symptom management apparatus 30 sets the symptom recording apparatus 20 so as to execute the symptom management according to the determined symptom management method by transmitting the information indicating the symptom management method to the symptom recording apparatus 20. Therefore, according to the present embodiment, it is possible to support the symptom management according to the individual backgrounds of the patient.

As to be described later, the control unit 31 of the symptom management apparatus 30 may acquire information indicating an implementation situation of the symptom management on the set symptom recording apparatus 20, and may update, based on the implementation situation of the symptom management on the symptom recording apparatus 20, the symptom management method according to the symptom recording apparatus 20 executes the symptom management. When the symptom management method is updated, by setting the symptom recording apparatus 20 such that the symptom recording apparatus 20 executes the symptom management according to the updated symptom management method, it is possible to automatically adjust the symptom management method according to an actual implementation situation of the symptom management.

Operation Example

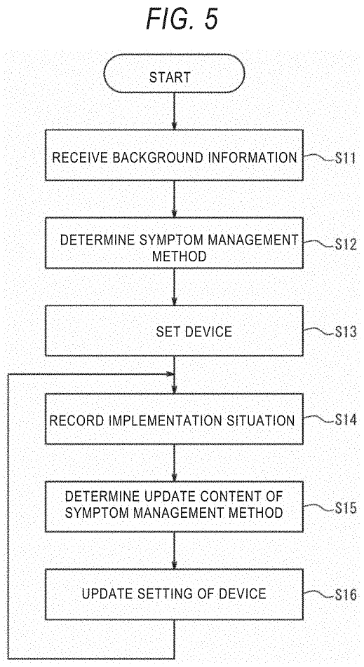

An operation example of the system 10 including the symptom recording apparatus 20 and the symptom management apparatus 30 according to the present embodiment will be described with reference to FIG. 5. FIG. 5 is a flowchart illustrating the operations of the symptom recording apparatus 20 and the symptom management apparatus 30. The operations performed are executed under the control of the control unit 21 of the symptom recording apparatus 20 or the control unit 31 of the symptom management apparatus 30. These operations correspond to an information processing method according to the present embodiment.

In S11, the symptom management apparatus 30 acquires the background information by receiving the background information indicating at least one of the physical condition and the living environment of the patient. The background information may include information indicating at least one of the pathological condition, the co-morbidity, the cognitive function, the depression tendency, the region characteristic, and the family circumstance of the patient. The input of the background information may be performed by receiving the background information from a medical device via a network in the medical institution, and may be performed by an operation of a user such as a doctor, a laboratory technician, or a nurse. Alternatively, the information indicating the implementation situation of the symptom management may be received from the symptom recording apparatus 20, and the information may be acquired as the background information.

FIG. 6 shows enlarged information indicating the line graph 51 that is an example of the background information and the line graph 52 that is an example of the background information or the implementation situation of the symptom management, which are included in the screen example of FIG. 4. The line graph 51 shows an assessment result investigated and measured in the medical institution. In an example of FIG. 6, the line graph 51 shows a medication adherence, an intake calorie, a salinity intake amount, the exercise amount, an exercise tolerance, a muscle strength, the cognitive function, and the depression. The line graph 52 shows an assessment result investigated and measured in the medical institution. The line graph 52 shows the information investigated and measured by the patient with a line graph.

The line graph 52 shows the investigated and measured results of the body weight, the blood pressure, the lower leg edema, the orthopnea, the difficulty breathing, the exercise amount, a nutrient intake amount, and the salinity. In an example of the line graph 52, investigations and measurements on all the management items are performed on May 1, but no investigation and measurement on the nutrient intake amount are performed on April 1. The control unit 31 can determine the symptom management method according to not only values of the investigated and measured management items, but also whether the investigations and the measurements are actually performed.

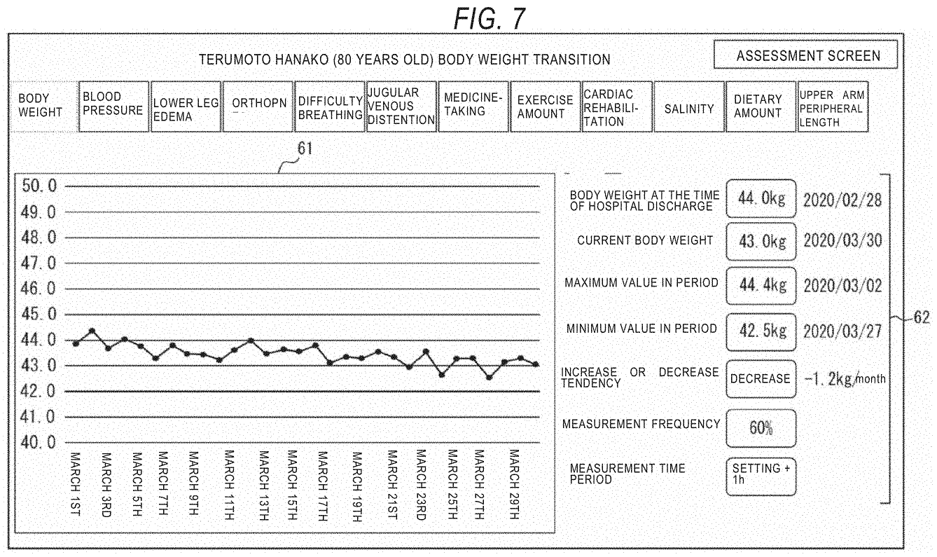

FIG. 7 is a screen example showing a body weight transition of the patient, which is an example of the background information or the implementation situation of the symptom management. A line graph 61 showing a body weight transition of the patient in a certain period (for example, one month). A display example 62 of a body weight at the time of hospital discharge, a current body weight, a maximum value in the period, a minimum value in the period, an increase or decrease tendency, a measurement frequency, and a measurement time period. In S15 to be described later, the control unit 31 may change the measurement time period displayed on the symptom recording apparatus 20 when a frequency of the measurement actually performed by the patient is relatively low and a deviation in the measurement time period is relatively large as compared with the symptom management method determined by the symptom management apparatus 30. For example, in display example 62 of FIG. 7, “60%” of the “measurement frequency” indicates that the number of body weight measurements actually performed by the patient is only 60% of the number of body weight measurements determined by the symptom management apparatus 30. “Setting+1 h” of the “measurement time period” indicates that the time period during which the measurement is actually performed is one hour later than the time period that is determined by the symptom management apparatus 30 and that is set in the symptom recording apparatus 20. In such a case, the symptom management apparatus 30 can change the measurement time period so as to be delayed by one hour. In this way, it is possible to expect an improvement in the measurement frequency by matching a measurement time to be presented with an actual measurement state on a patient side.

In S12, the symptom management apparatus 30 determines the symptom management method to be set on the symptom recording apparatus 20 based on the acquired background information. The determination of the symptom management method can be performed according to a predetermined rule. Such a rule indicates a relationship (i.e., corresponding relationship) between the at least one of the physical condition and the living environment of the patient and the at least one of the management item to be managed at the time of managing the symptom of the patient and the method for providing information to the patient.

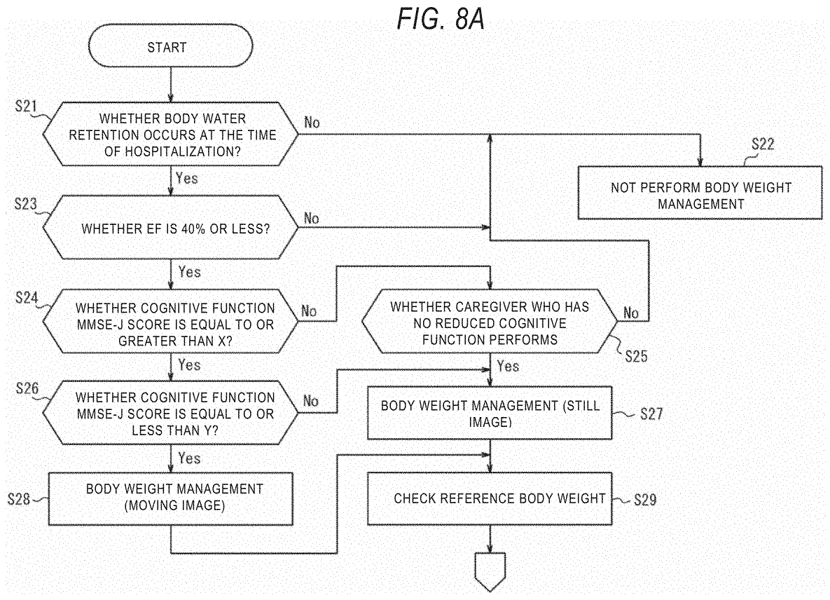

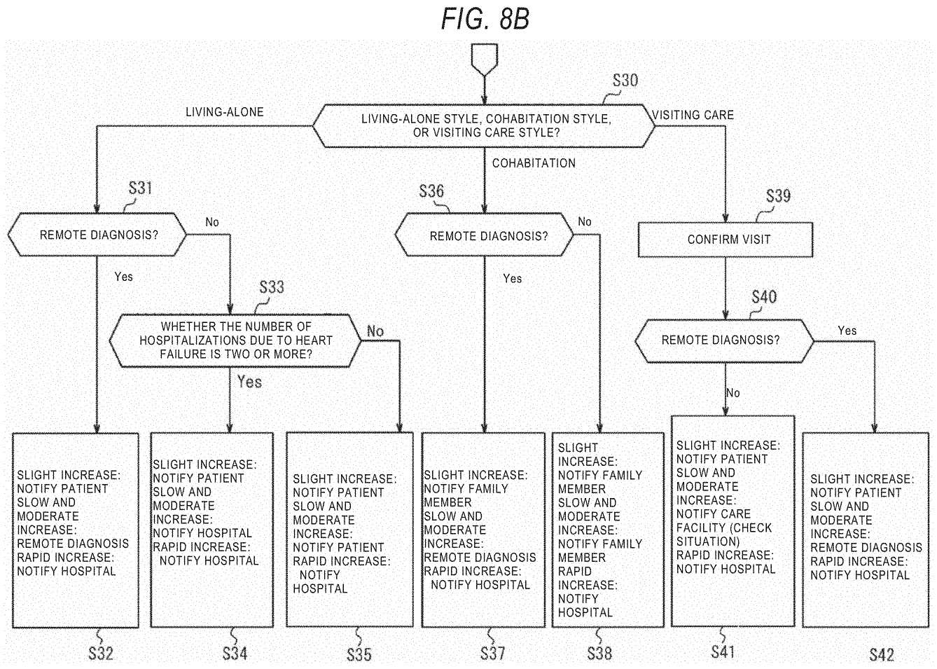

An operation example of determining the symptom management method according to the predetermined rule will be described with reference to FIGS. 8A and 8B. FIGS. 8A and 8B are flowcharts illustrating an example of a rule for determining a symptom management method related to the body weight management.

In S21, the symptom management apparatus 30 determines whether a body water retention, which indicates the presence of congestion in the body of the patient, occurs at the time of hospitalization. When the body water retention occurs (Yes in S21), a process proceeds to S23, and when no body water retention occurs (i.e., water retention in the body does not occur) (No in S21), the process proceeds to S22.

In S22, the symptom management apparatus 30 determines that the body weight management is not performed, and ends the process. In S23, the symptom management apparatus 30 determines whether an ejection fraction (EF) is, for example, 40% or less. When the ejection fraction is, for example, 40% or less (Yes in S23), the process proceeds to S24, and when the ejection fraction is more than 40% (i.e., greater than 40%) (No in S23), the process proceeds to S22.

In S24, the symptom management apparatus 30 can determine whether a cognitive function MMSE-J score is equal to or greater than a predetermined value X. The MMSE-J is an abbreviation of Mini Mental State Examination-Japanese. The X is a reference value indicating that the patient himself/herself has a minimum cognitive function necessary for performing the body weight management by himself/herself. When the score is equal to or greater than the X, that is, when the patient himself/herself has the minimum cognitive function necessary for performing the body weight management by himself/herself (Yes in S24), the process proceeds to S26, and when the score is less than X (No in S24), the process proceeds to S25.

In S25, the symptom management apparatus 30 determines whether a caregiver who has no problem in the cognitive function can measure the body weight of the patient. When the measurement can be performed (Yes in S25), the process proceeds to S27, and when the measurement cannot be performed (No in S25), the process proceeds to S22.

In S26, the symptom management apparatus 30 determines whether the cognitive function MMSE-J score is equal to or less than a predetermined value Y. The Y is a value larger than the X. Although a patient whose cognitive function MMSE-J score is equal to or greater than X and equal to or less than Y has a cognitive function that only allows the patient to perform the body weight management by himself/herself, but requires a detailed explanation of a moving image. On the other hand, a patient whose cognitive function MMSE-J score exceeds the Y can perform the body weight management by himself/herself with only an explanation of a still image. When the cognitive function MMSE-J score is equal to or less than the Y (Yes in S26), the process proceeds to S28, and when the score exceeds the Y (No in S26), the process proceeds to S27.

In S27, the symptom management apparatus 30 determines that the explanation of the body weight management is displayed by using a still image from the output unit 25 of the symptom recording apparatus 20. In S28, the symptom management apparatus 30 determines that the explanation of the body weight management is displayed by using a moving image from the output unit 25 of the symptom recording apparatus 20. Since the moving image is relatively easier to understand than the still image, the body weight management can be described in an easily understandable manner even for the patient with the lowered cognitive function who can perform the body weight management by himself/herself. A voice, a vibration, or both of the voice and the vibration may be output as appropriate depending on an application.

In S29, the symptom management apparatus 30 checks a reference body weight. In S30 and subsequent steps, the symptom management apparatus 30 performs a process of determining, based on background information such as a living style, the presence or absence of remote diagnosis, and a hospitalization history, which person is to be notified when the body weight of the patient changes as compared with the reference body weight. In S30, the symptom management apparatus 30 determines whether the living style of the patient is a living-alone style, a cohabitation style, or a visiting care style. In a case of the living-alone style (living-alone in S30), the process proceeds to S31. In a case of the cohabitation style (cohabitation in S30), the process proceeds to S36. In a case of the visiting care style (visiting care in S30), the process proceeds to S39.

In S31, the symptom management apparatus 30 determines whether the remote diagnosis is performed on the patient. When the remote diagnosis is performed (Yes in S31), the process proceeds to S32, and when the remote diagnosis is not performed (No in S31), the process proceeds to S33.

In S32, when an increase in the body weight is a slight change as compared with the reference body weight, the symptom management apparatus 30 determines to notify the patient of the change in the body weight of the patient. When the increase in the body weight is a slow and moderate increase, the symptom management apparatus 30 determines to perform the remote diagnosis. When the increase in the body weight is a rapid increase, the symptom management apparatus 30 determines to notify a hospital of the increase. Then, the symptom management apparatus 30 ends the process.

In S33, the symptom management apparatus 30 determines whether the number of past hospitalizations due to the heart failure of the patient, for example, is two or more. When the number of past hospitalizations, for example, is two or more (Yes in S33), the process proceeds to S34, and when the number of past hospitalizations, for example, is less than two (No in S33), the process proceeds to S35. In S34, when the increase in the body weight is a slight change as compared with the reference body weight, the symptom management apparatus 30 determines to notify the patient of the change, and when the increase in the body weight is a slow and moderate increase, or the increase in the body weight is a rapid increase, the symptom management apparatus 30 determines to notify the hospital of the increase. Then, the symptom management apparatus 30 ends the process. In S35, when the increase in the body weight is a slight change as compared with the reference body weight, or when the increase in the body weight is a slow and moderate increase, the symptom management apparatus 30 determines to notify the patient of the increase. When the increase in the body weight is a rapid increase, the symptom management apparatus 30 determines to notify the hospital of the increase. Then, the symptom management apparatus 30 ends the process.

In S36, the symptom management apparatus 30 determines whether the remote diagnosis is performed on the patient. When the remote diagnosis is performed (Yes in S36), the process proceeds to S37, and when the remote diagnosis is not performed (No in S36), the process proceeds to S38. In S37, when the increase in the body weight is a slight change as compared with the reference body weight, the symptom management apparatus 30 determines to notify the family member of the patient of the change. When the increase in the body weight is a slow and moderate increase, the symptom management apparatus 30 determines to perform the remote diagnosis. When the increase in the body weight is a rapid increase, the symptom management apparatus 30 determines to notify the hospital of the increase. Then, the symptom management apparatus 30 ends the process. In S38, when the increase in the body weight is a slight change as compared with the reference body weight, or when the increase in the body weight is a slow and moderate increase, the symptom management apparatus 30 determines to notify the family member of the increase. When the increase in the body weight is a rapid increase, the symptom management apparatus 30 determines to notify the hospital of the increase. Then, the symptom management apparatus 30 ends the process.

In S39, the symptom management apparatus 30 confirms a visit date of the caregiver. In S40, the symptom management apparatus 30 determines whether the remote diagnosis is performed on the patient. When the remote diagnosis is not performed (No in S40), the process proceeds to S41, and when the remote diagnosis is performed (Yes in S40), the process proceeds to S42. In S41, when the increase in the body weight is a slight change as compared with the reference body weight, the symptom management apparatus 30 determines to notify the patient of the change. When the increase in the body weight is a slow and moderate increase, the symptom management apparatus 30 determines to notify a care facility of the increase, and to check a situation. When the increase in the body weight is a rapid increase, the symptom management apparatus 30 determines to notify the hospital of the increase. Then, the symptom management apparatus 30 ends the process. In S42, when the increase in the body weight is a slight change as compared with the reference body weight, the symptom management apparatus 30 determines to notify the patient of the change. When the increase in the body weight is a slow and moderate increase, the symptom management apparatus 30 determines to perform the remote diagnosis. When the increase in the body weight is a rapid increase, the symptom management apparatus 30 determines to notify the hospital of the increase. Then, the symptom management apparatus 30 ends the process.

As described above, in S30 and subsequent steps, the symptom management apparatus 30 performs the process of determining, based on the background information such as the living style, the presence or absence of remote diagnosis, and the hospitalization history, which person is to be notified when the body weight of the patient changes as compared with the reference body weight. Therefore, it is possible to optimize the management for an individual patient according to the background information of the patient, and it is possible to accurately perform the symptom management without omission and waste.

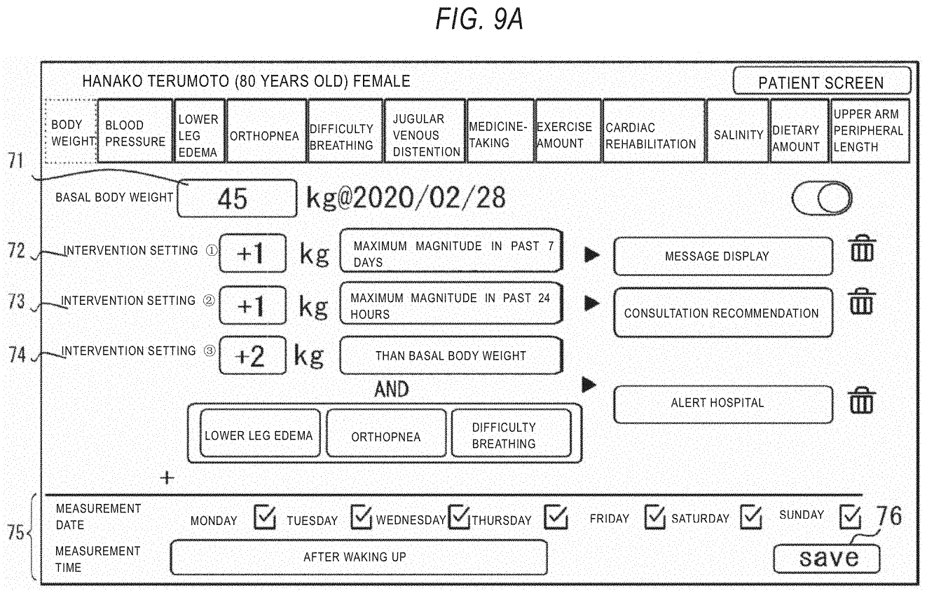

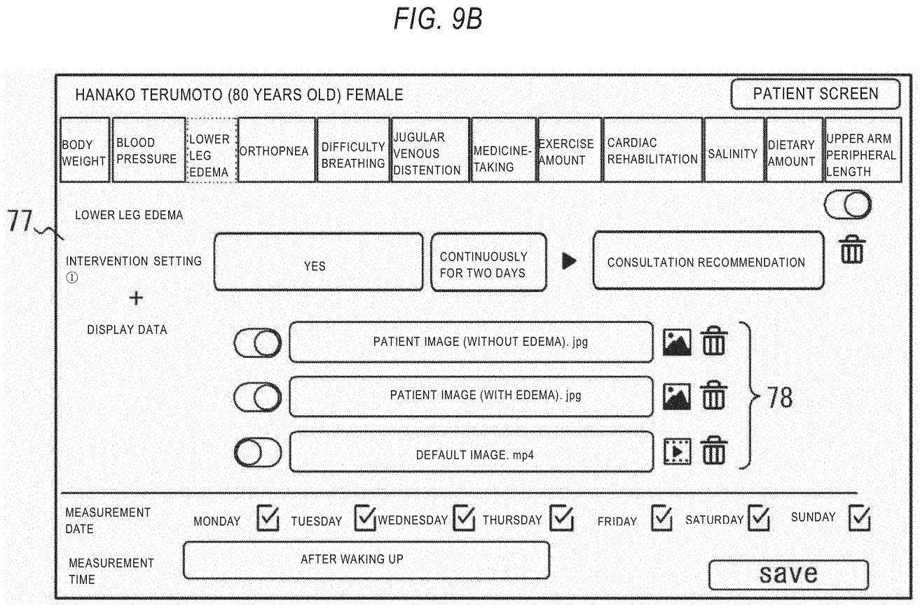

In FIGS. 8A and 8B, an example is described in which the symptom management method for the body weight management serving as a management item is determined, and the symptom management method for other management items can also be determined based on the background information of the patient. FIGS. 9A and 9B show examples in which a management item related to the intervention for the patient is determined based on the background information. FIG. 9A shows an intervention corresponding to a body weight change, and FIG. 9B shows an intervention corresponding to the lower leg edema.

In FIG. 9A, a basal body weight 71 (45 kg) of the patient “Hanako Terumoto” is shown. An intervention of displaying a predetermined message 72 is shown when a maximum magnitude of the body weight change in the past 7 days exceeds 1 kg. An intervention of performing a consultation recommendation 73 is shown when a maximum magnitude of the body weight change in the past 24 hours exceeds 1 kg. An intervention of alerting the hospital 74 is shown when the body weight increases by 2 kg or more as compared with the basal body weight, and the physical condition corresponds to the lower leg edema, the orthopnea, or the difficulty breathing. The body weight of the patient 75 is measured after waking up on Monday, Tuesday, Wednesday, Thursday, Friday, Saturday, and Sunday. Although 71 to 75 are automatically determined based on the background information, the user such as a doctor can correct and update a content of each item by an operation. For example, the measurement time period may be changed for each day of the week. In addition, the measurement time period may be a specific time such as 8 o'clock. After the correction, when the user selects a “save” button 76, the content after correction is determined as the symptom management method.

In FIG. 9B, an intervention of performing a consultation recommendation 77 is shown when the lower leg edema develops continuously for two days. In addition, a patient image (without edema) and a patient image (with edema) 77 can be displayed when the symptom is confirmed. FIG. 9B also shows that the lower leg edema can be, for example, measured after waking up on Monday, Tuesday, Wednesday, Thursday, Friday, Saturday, and Sunday. Although items in FIG. 9B are also automatically determined based on the background information, the user such as a doctor can correct and update a content of each item by an operation. After the correction, when the user selects the “save” button, the content after correction is determined as the symptom management method. As described above, when the operation for updating the symptom management method is performed by the user after the symptom management method is determined by the symptom management apparatus 30, the symptom management apparatus 30 may set the symptom recording apparatus 20 so as to execute the symptom management according to the updated symptom management method. Accordingly, it is possible to perform a more appropriate symptom management by reflecting the determination of the user such as a doctor in the selection of the symptom management method.

The description returns to FIG. 5. When the symptom management method is determined in S12, in S13, the symptom management apparatus 30 performs a process of setting the determined symptom management method to the symptom recording apparatus 20 (also referred to as a device). In the present embodiment, the symptom management apparatus 30 sets the symptom recording apparatus 20 so as to execute the symptom management according to the symptom management method by transmitting the information indicating the symptom management method to the symptom recording apparatus 20, but the present invention is not limited thereto. For example, the symptom management apparatus 30 may record the information indicating the symptom management method in a recording medium such as a USB memory, transmit the information to the patient side, and set the symptom recording apparatus 20 on the patient side by reading the information into the symptom recording apparatus 20.

In S14, the symptom recording apparatus 20 performs a display for performing the symptom management corresponding to the patient according to the symptom management method set by the symptom management apparatus 30, performs the symptom management, and records an implementation situation including a measurement result and the like.

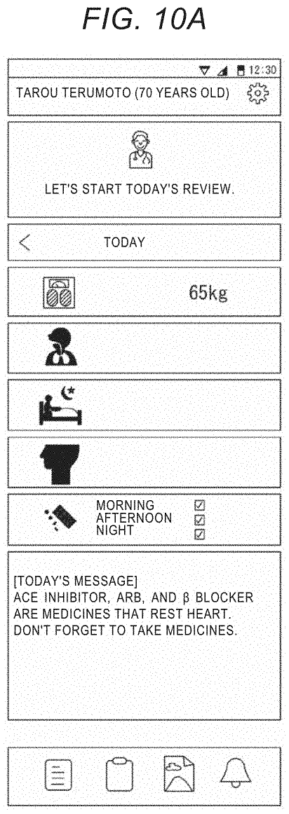

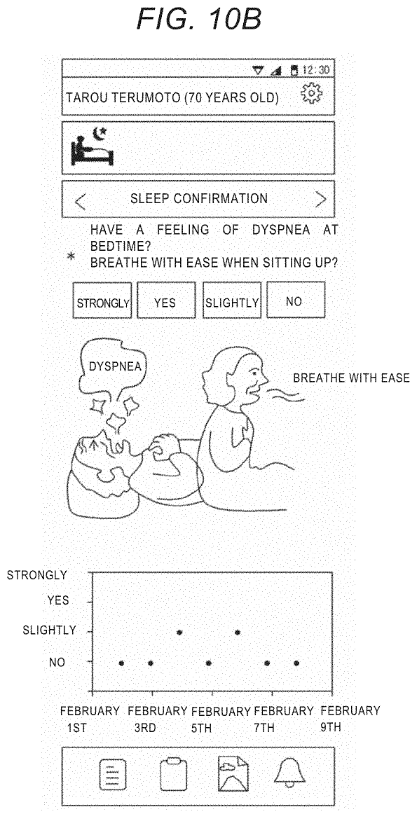

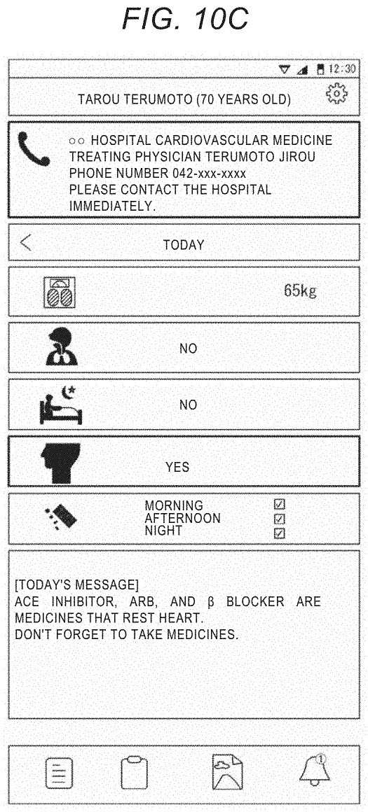

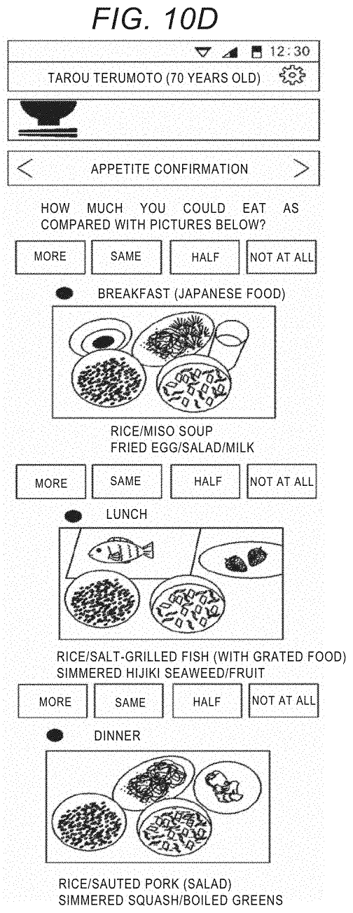

FIGS. 10A, 10B, 10C, and 10D are diagrams illustrating examples of a display screen of the symptom recording apparatus 20. FIG. 10A is an initial screen of the symptom recording apparatus 20. A message on medicine-taking of pharmaceuticals corresponding to a condition of the patient is displayed on a lower part of the screen. FIG. 10B is an example of a screen for recording a symptom of the orthopnea. FIG. 10C shows an example of an initial screen on which a notification of immediately contacting the hospital is displayed according to a condition of the patient “Tarou Terumoto”. FIG. 10D shows an example of a screen for confirming an appetite. The screen of the symptom recording apparatus 20 can be customized and displayed for each patient according to the symptom management method determined based on the background information. A voice, a vibration function, or both of the voice and the vibration function, for example, may be used based on an ability of the patient or the like. In addition, although FIG. 10C shows the example of the screen on which the notification of immediately contacting the hospital is displayed, for example, a reservation such as an online medical examination may be performed according to an environment. In addition, although an image of Japanese food is displayed in FIG. 10D, an image, for example, such as western food may be displayed according to a dietary life of the patient.

According to the displays as shown in FIGS. 10A to 10D, the patient, the family member of the patient, the caregiver or the like may measure examination items and input results of the examination items to the symptom recording apparatus 20. The symptom recording apparatus 20 transmits the information indicating the received implementation situation of the symptom management to the symptom management apparatus 30. The symptom management apparatus 30 records the received implementation situation of the symptom management into the storage unit 32.

The description returns to FIG. 5. In S15, the symptom management apparatus 30 determines an update content of the symptom management method based on the implementation situation of the symptom management. The determination of the update content of the symptom management method in S15 can be performed according to a predetermined rule as in S12. Such a rule indicates a relationship (i.e., corresponding relationship) between the implementation situation of the symptom management and the at least one of the management item to be managed at the time of managing the symptom of the patient and the method for providing information to the patient.

For example, as described above with reference to FIG. 7, the control unit 31 may change the measurement time period displayed on the symptom recording apparatus 20 when the frequency of the measurement actually performed by the patient is low and the deviation in the measurement time period is large as compared with the symptom management method determined by the symptom management apparatus 30. Specifically, for example, it is assumed that the measurement of the body weight is set, for example, to be performed at 8 o'clock every morning, and such a matter is displayed on the symptom recording apparatus 20, but days when the measurement is actually performed in one month are less than or equal to half of a month, and the measurement time period is, for example, 9 o'clock in most of the days when the measurement is performed. In such a case, the measurement frequency can be expected to be improved by changing the measurement time from 8 o'clock to 9 o'clock. Further, the measurement time period may be analyzed by distinguishing between a weekday and a holiday, or the measurement time period may be analyzed for each day of the week, and a result of the measurement time period may be reflected in the update of the symptom management method.

In addition, for example, it can be assumed that the symptom confirmation of the lower leg edema is notified to the patient only by displaying text information, and most of answer contents of the patient are “unknown”. In such a case, a confirmation method for the symptom of the lower leg edema can be changed from a display of the text information to an output of a moving image display, a voice, or both of the moving image display and the voice. As a result, it is possible to notify the patient of the confirmation method for the symptom in a relatively easily understandable manner, and to expect the appropriate implementation of the symptom confirmation.

Further, for example, regarding the salinity intake amount, it is assumed that previously, the salinity intake amount is set to the management items to reflect an assessment result that the salinity is slightly excessive, and thereafter, although the salinity intake amount decreases, a decreasing tendency is observed in the body weight. In such a case, the salinity intake amount can be excluded from the management items, and the dietary amount can be used as a management item. As a result, it is possible to perform the symptom management according to a change in a lifestyle habit of the patient.

In S16, the symptom management apparatus 30 sets the update content of the symptom management method determined in S15 to the symptom recording apparatus 20. The setting can be performed, for example, by transmitting the information indicating the update content of the symptom management method to the symptom recording apparatus 20. When an operation for further updating the symptom management method is performed by the user after the determination of the update content in S15, the symptom management apparatus 30 may set the symptom recording apparatus 20 so as to execute the symptom management according to the symptom management method reflecting the operation of the user. Accordingly, it is possible to perform a more appropriate symptom management by reflecting the determination of the user such as a doctor in the selection of the symptom management method. When the process in S16 ends, the process returns to S14.

As described above, with the configuration according to the present embodiment, it is possible to optimize self-care items for the individual patient, and thus it is possible to accurately perform the symptom management without omission and waste.

In the above-described example, in S12 of FIG. 5, the symptom management method is determined based on the background information according to the predetermined rule. The predetermined rule indicates the relationship (i.e., corresponding relationship) between the at least one of the physical condition and the living environment of the patient and the at least one of the management item to be managed at the time of managing a symptom of the patient and the method for providing information to the patient. The determination of the update content of the symptom management method in S15 is also performed according to the predetermined rule. However, the determination of the symptom management method or the update content of the symptom management method may be performed by using a prediction model that reflects a result of machine learning based on a previous record, instead of being performed according to the predetermined rule. That is, for at least one sample patient including at least one of: a target patient who is a patient as a subject of symptom management, and at least one another patient, the symptom management apparatus 30 creates and acquires, in advance, a prediction model which is created through machine learning by using, as training data, in a symptom management performed in the past for the sample patient, at least one of background information of the sample patient, a symptom management method of the symptom management performed in the past, and an implementation situation of the symptom management performed in the past, and a health condition of the sample patient after the symptom management performed in the past is performed. As such training data, it is possible to use data of many patients (sample patients) collected in a certain range such as a region, a hospital, or a diagnosis-and-treatment department. The health condition of the sample patient after the symptom management is performed is, for example, a rehospitalization probability for 30 days, a rehospitalization probability for 1 year, a 3-year survival rate, a 5-year survival rate, a continuation rate, cardiac function, the muscle strength, the cognitive function, or the like. Then, when determining a symptom management method for the target patient based on background information of the target patient, the symptom management apparatus 30 predicts, using such a prediction model, a health condition of the target patient based on at least one of the background information of the target patient, the symptom management method for the target patient, and the implementation situation of the symptom management for the target patient. Further, the symptom management apparatus 30 determines the symptom management method for the target patient or an update content of the symptom management apparatus 30 based on the predicted health condition of the target patient. As described above, by determining the symptom management method or the update content of the symptom management method based on the prediction model created through the machine learning, it is possible to perform setting specialized for a region or a hospital based on data stored in the region or the hospital.

Here, an initial candidate of the symptom management method may be set based on a rule, and a plurality of candidates may be created based on the prediction model by adding an achievable change to the initial candidate at a stage where data related to a certain record is collected. Then, the prediction may be performed by inputting implementation information and the background information, and selections by the doctor may be supported by presenting respective scores. Alternatively, regarding items whose total score or respective scores do not fall below a predetermined threshold, items having a high total score may be ranked and presented in the order from the top. In addition, by referring to how much the score changes due to the correction of the doctor, learning an amount of change of the score due to the correction of the doctor, and reflecting the amount of change in the determination of the symptom management method, it is possible to present a symptom management method according to a diagnostic policy of an individual doctor.

The determination of the symptom management method or the update content of the symptom management method may be determined based on a rule predetermined for contents of all the symptom management methods, or may be determined based on a prediction model created through the machine learning for the contents of all the symptom management methods. Alternatively, the determination of the symptom management method or the update content of the symptom management method may be determined based on a rule predetermined for contents of a part of the symptom management methods, and based on a prediction model created through the machine learning for contents of the remaining symptom management methods. For example, the selection of the management item may be determined based on the prediction model created through the machine learning, and the intervention may be determined based on the predetermined rule. When the determination based on the prediction model created through the machine learning is limited to a part of the symptom management methods, it is possible to create the prediction model as long as the contents of a part of the symptom management methods are present, and thus it is possible to determine the symptom management method or the update content of the symptom management method based on the prediction model even an amount of stored data is relatively small.

In the embodiment described above, an example is described in which both of the determination of the symptom management method in S12 of FIG. 5 and the determination of the update content of the symptom management method in S15 are performed by the symptom management apparatus 30, and the user can update the content of the determination as appropriate. However, the process of at least one of S12 and S15 may be performed by the user instead of the symptom management apparatus 30 according to an application or a purpose.

The present disclosure is not limited to the above-described embodiment. For example, a plurality of blocks described in the block diagram may be integrated, or one block may be divided. Instead of executing a plurality of steps described in a flowchart in time series according to the description, the steps may be executed in parallel or in a different order according to the processing capability of the device that executes each step or as necessary. In addition, modifications can be made without departing from a gist of the present disclosure.

The detailed description above describes embodiments of an information processing apparatus, an information processing system, an information processing method, and a computer program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An information processing apparatus, comprising:
a control unit configured to:
determine, based on background information indicating one or more of a physical condition and a living environment of a patient, a symptom management method that includes one or more of: a management item to be managed at the time of managing a symptom of the patient, and a method for providing information to the patient;
set a terminal device such that the terminal device is configured to execute a symptom management according to the symptom management method;
acquire information indicating an implementation situation of the symptom management on the set terminal device;
update, based on the implementation situation of the symptom management on the terminal device, the symptom management method according to which the terminal device is configured to execute the symptom management; and
when the symptom management method is updated, set the terminal device such that the terminal device is configured to execute a symptom management according to the updated symptom management method.

2. The information processing apparatus according to claim 1, wherein the control unit is configured to set the terminal device so as to execute the symptom management according to the symptom management method by transmitting information indicating the symptom management method to the terminal device.

3. The information processing apparatus according to claim 1, wherein when an operation for updating the symptom management method is performed by a user, the control unit is configured to set the terminal device such that the terminal device executes the symptom management according to the updated symptom management method.

4. The information processing apparatus according to claim 1, wherein according to a rule indicating a relationship between the one or more of the physical condition and the living environment of the patient and the one or more of the management item to be managed at the time of managing the symptom of the patient and the method for providing information to the patient, the control unit is configured to determine the symptom management method based on the background information.

5. The information processing apparatus according to claim 1, further comprising:
at least one sample patient, the at least one sample patient includes one or more of: a target patient that is the patient, and at least one other patient; and
wherein the control unit is configured to acquire a prediction model which is created through machine learning by using, as training data, in a symptom management performed in the past for the at least one sample patient, one or more of background information of the at least one sample patient, a symptom management method of the symptom management performed in the past, and an implementation situation of the symptom management performed in the past, and a health condition of the sample patient after the symptom management performed in the past is performed; and
when determining a symptom management method for the target patient based on the background information of the target patient, the control unit is configured to predict a health condition of the target patient based on one or more of the background information of the target patient, the symptom management method for the target patient, and the implementation situation of the symptom management for the target patient by using the prediction model, and to determine a symptom management method for the target patient based on the predicted health condition of the target patient.

6. The information processing apparatus according to claim 1, wherein the background information includes information indicating one or more of a pathological condition, a co-morbidity, a cognitive function, a depression tendency, a region characteristic, and a family circumstance of the patient.

7. The information processing apparatus according to claim 1, wherein the symptom management method includes one or more of a symptom management item, a method for inputting a symptom to the terminal device, a method for providing information from the terminal device, and an intervention method for the patient.

8. An information processing system comprising:
the information processing apparatus according to claim 1; and
the terminal device.

9. An information processing method, comprising:
determining, by a control unit, based on background information indicating one or more of a physical condition and a living environment of a patient, a symptom management method that includes one or more of: a management item to be managed at the time of managing a symptom of the patient, and a method for providing information to the patient;
setting, by the control unit, a terminal device such that the terminal device executes a symptom management according to the symptom management method;
acquiring, by the control unit, information indicating an implementation situation of the symptom management on the set terminal device;
updating, by the control unit, based on the implementation situation of the symptom management on the terminal device, the symptom management method according to which the terminal device executes the symptom management; and
when the symptom management method is updated, setting, by a control unit, the terminal device such that the terminal device executes a symptom management according to the updated symptom management method.

10. The information processing method according to claim 9, further comprising:
setting the terminal device so as to execute the symptom management according to the symptom management method by transmitting information indicating the symptom management method to the terminal device.

11. The information processing method according to claim 9, wherein when an operation for updating the symptom management method is performed by a user, further comprising:
setting the terminal device such that the terminal device executes the symptom management according to the updated symptom management method.

12. The information processing method according to claim 9, wherein according to a rule indicating a relationship between the one or more of the physical condition and the living environment of the patient and the one or more of the management item to be managed at the time of managing the symptom of the patient and the method for providing information to the patient, further comprising:
determining the symptom management method based on the background information.

13. The information processing method according to claim 9, further comprising:
acquiring a prediction model which is created through machine learning by using, as training data, in a symptom management performed in the past for at least one sample patient, the at least one sample patient includes one or more of a target patient that is the patient and at least one other patient, and wherein the training data includes:
one or more of background information of the at least one sample patient, a symptom management method of the symptom management performed in the past, and an implementation situation of the symptom management performed in the past; and
a health condition of the at least one sample patient after the symptom management performed in the past is performed; and
when determining a symptom management method for the target patient based on the background information of the target patient, predicting a health condition of the target patient based on one or more of the background information of the target patient, the symptom management method for the target patient, and the implementation situation of the symptom management for the target patient by using the prediction model, and determining a symptom management method for the target patient based on the predicted health condition of the target patient.

14. The information processing method according to claim 9, wherein the background information includes information indicating one or more of a pathological condition, a co-morbidity, a cognitive function, a depression tendency, a region characteristic, and a family circumstance of the patient.

15. The information processing method according to claim 9, wherein the symptom management method includes one or more of a symptom management item, a method for inputting a symptom to the terminal device, a method for providing information from the terminal device, and an intervention method for the patient.

16. A non-transitory computer-readable medium (CRM) storing computer program code executed by a computer processor that executes a process comprising:
determining, based on background information indicating one or more of a physical condition and a living environment of a patient, a symptom management method that includes one or more of: a management item to be managed at the time of managing a symptom of the patient, and a method for providing information to the patient;
setting a terminal device such that the terminal device executes a symptom management according to the symptom management method;
acquiring information indicating an implementation situation of the symptom management on the set terminal device;
updating, based on the implementation situation of the symptom management on the terminal device, the symptom management method according to which the terminal device executes the symptom management; and
when the symptom management method is updated, setting the terminal device such that the terminal device executes a symptom management according to the updated symptom management method.

17. The computer-readable medium according to claim 16, further comprising:
setting the terminal device so as to execute the symptom management according to the symptom management method by transmitting information indicating the symptom management method to the terminal device.

18. The computer-readable medium according to claim 16, wherein when an operation for updating the symptom management method is performed by a user, further comprising:
setting the terminal device such that the terminal device executes the symptom management according to the updated symptom management method.

19. The computer-readable medium according to claim 16, wherein according to a rule indicating a relationship between the one or more of the physical condition and the living environment of the patient and the one or more of the management item to be managed at the time of managing the symptom of the patient and the method for providing information to the patient, further comprising:
determining the symptom management method based on the background information.

20. The computer-readable medium according to claim 16, further comprising:
acquiring a prediction model which is created through machine learning by using, as training data, in a symptom management performed in the past for at least one sample patient, the at least one sample patient includes one or more of a target patient that is the patient and at least one other patient, the training data including:
one or more of background information of the at least one sample patient, a symptom management method of the symptom management performed in the past, and an implementation situation of the symptom management performed in the past; and
a health condition of the at least one sample patient after the symptom management performed in the past is performed; and
when determining a symptom management method for the target patient based on the background information of the target patient, predicting a health condition of the target patient based on one or more of the background information of the target patient, the symptom management method for the target patient, and the implementation situation of the symptom management for the target patient by using the prediction model, and determining a symptom management method for the target patient based on the predicted health condition of the target patient.
